(19) United States
(12) Patent Application Publication
(10) Pub. No.: US 2024/0240231 A1
(43) Pub. Date: Jul. 18, 2024

(54) DETECTION DEVICE AND DETECTION METHOD

(71) Applicant: SHIMADZU CORPORATION, Kyoto (JP)

(72) Inventor: Hiroto TANAKA, Kyoto (JP)

(73) Assignee: SHIMADZU CORPORATION, Kyoto (JP)

(21) Appl. No.: 18/401,532

(22) Filed: Dec. 31, 2023

(30) Foreign Application Priority Data
Jan. 13, 2023 (JP) 2023-003703

(51) Int. Cl. C12Q 1/68

(52) U.S. Cl. CPC C12Q 1/68

(57) ABSTRACT

A detection device includes a CPU and a memory. CPU sets a boundary point at which a melting curve is divided into a front region and a rear region, sets a start point and an end point, calculates an approximate straight line by a least squares method, and repeats setting processing of the approximate straight line. CPU sets a first straight line in the front region, sets a second straight line in the rear region, and detects, as a melting temperature, a temperature of a nucleic acid corresponding to an intersection between a melting curve and a line connecting intermediate values of absorbance of the nucleic acid at which a temperature of the nucleic acid is the same on the first straight line and the second straight line.

### CROSS REFERENCE TO RELATED APPLICATIONS

This nonprovisional application is based on Japanese Patent Application No. 2023-003703 filed on Jan. 13, 2023, with the Japan Patent Office, the entire contents of which are hereby incorporated by reference.

### BACKGROUND OF THE INVENTION

### Field of the Invention

The present disclosure relates to a detection device and a detection method for detecting a melting temperature of a nucleic acid.

### Description of the Background Art

There is a melting temperature (hereinafter, also referred to as Tm value) as an index indicating stability of a nucleic acid such as deoxyribonucleic acid (DNA). The nucleic acid has a double-stranded structure. The double-stranded structure of the nucleic acid is gradually separated by increasing the temperature of a solution in which the nucleic acid is dissolved, and is completely separated into a single-stranded structure in a high-temperature solution. When the nucleic acid changes from a double-stranded structure to a single-stranded structure, absorbance of ultraviolet light around a wavelength of 260 μm greatly increases. Since the nucleic acid has such characteristics, when a graph of a melting curve with a horizontal axis as temperature and a vertical axis as absorbance is created, a region where the graph changes rapidly appears. The Tm value is a temperature at which the ratio of the single-stranded structure and the double-stranded structure in such a region is equal.

Japanese Patent Laying-Open No. 2003-121396 discloses a device capable of calculating absorbance of DNA with high accuracy. Thus, in Japanese Patent Laying-Open No. 2003-121396, a graph of a melting curve with high accuracy can be created.

### SUMMARY OF THE INVENTION

The Tm value can be obtained, for example, by the following method (hereinafter, also referred to as “median method”). In the median method, a start point and an end point are set in a front region (a region of a double-stranded structure) and a rear region (a region of a single-stranded structure) in a graph of a nucleic acid, and a straight line connecting the two points is drawn. The median method is a method of setting, as the Tm value, a temperature of the nucleic acid at an intersection of a melting curve and a line connecting intermediate values of absorbance of the nucleic acid at which the temperature of the nucleic acid is the same on the two straight lines. Japanese Patent Laying-Open No. 2003-121396 does not disclose a detailed method of detecting the Tm value.

When the median method is used to calculate the Tin value, a user arbitrarily sets the start point and the end point. Therefore, even when the same data is used, the Tm value varies between the start point and the end point for each user, and thus an error may occur in the detection.

The present disclosure has been made to solve such a problem, and an object of the present disclosure is to provide a detection device and a detection method capable of eliminating a detection error of a melting temperature (Tm value) for each user.

A detection device of the present disclosure relates to a detection device that detects a melting temperature of a nucleic acid by using data indicating a relationship between a temperature of the nucleic acid and absorbance of the nucleic acid corresponding to ultraviolet light, the temperature and the absorbance being measured by an analysis device. The detection device includes a control device and a storage device that stores data from the analysis device. The data is represented by a melting curve in which the absorbance of the nucleic acid increases as the temperature of the nucleic acid increases when the data is plotted with the temperature of the nucleic acid on a horizontal axis and the absorbance of the nucleic acid on a vertical axis. The control device sets a boundary point dividing the data stored in the storage device into a front region and a rear region in accordance with the temperature of the nucleic acid when the data is expressed as the melting curve, sets a minimum temperature as a first start point and a maximum temperature as a first end point on first data defined by a predetermined number of data continuously changing in accordance with the temperature of the nucleic acid among the data in the front region, calculates an approximate straight line by a least squares method by using data in a range between the first start point and the first end point, while moving a range of the first data in a direction of the boundary point until the first end point reaches the boundary point, repeats setting processing of the approximate straight line using each of the first data after the moving, sets a maximum temperature as a second start point and a minimum temperature as a second end point on second data defined by a predetermined number of data continuously changing in accordance with the temperature of the nucleic acid among the data in the rear region, calculates an approximate straight line by the least squares method by using data in a range between the second start point and the second end point, while moving a range of the second data in a direction of the boundary point until the second end point reaches the boundary point, repeats setting processing of the approximate straight line using each of second data after the moving, sets a first straight line that maximizes K1×W1+1/r when a first constant preset on the basis of a plot interval of adjacent data in the first data is denoted by K1, a temperature difference between the first start point and the first end point of the first data is denoted by W1, and a gradient of the approximate straight line calculated from the first data is denoted by r, sets a second straight line that maximizes K2×W2+1/r when a second constant preset on the basis of a plot interval of adjacent data in the second data is denoted by K2, a temperature difference between the second start point and the second end point of the second data is denoted by W2, and a gradient of the approximate straight line calculated from the second data is denoted by r, and detects, as the melting temperature, the temperature of the nucleic acid corresponding to an intersection between the melting curve and a line connecting intermediate values of the absorbance of the nucleic acid at which the temperature of the nucleic acid is the same on the first straight line and the second straight line.

A detection method of the present disclosure relates to a detection method of detecting a melting temperature of a nucleic acid by using data indicating a relationship between a temperature of the nucleic acid and absorbance of the nucleic acid corresponding to ultraviolet light, the temperature and the absorbance being measured by an analysis device. The data is represented by a melting curve in which the absorbance of the nucleic acid increases as the temperature of the nucleic acid increases when the data is plotted with the temperature of the nucleic acid on a horizontal axis and the absorbance of the nucleic acid on a vertical axis. A method includes executing steps of recording data from an analysis device, setting a boundary point dividing the data stored into a front region and a rear region in accordance with the temperature of the nucleic acid when the data is expressed as the melting curve, setting a start point and an end point in order from a point at which the temperature of the nucleic acid is lower on the melting curve on a first number of data continuously changing in accordance with the temperature of the nucleic acid among the data in the front region, calculating an approximate straight line by a least squares method by using the first number of data in a range between the first start point and the first end point, repeating setting processing of the approximate straight line from the first number of data while moving the start point and the end point one by one in a direction of the boundary point until the end point reaches the boundary point, setting a start point and an end point in order from a point at which the temperature of the nucleic acid is higher on a second number of data continuously changing in accordance with the temperature of the nucleic acid among the data in the rear region, calculating an approximate straight line by the least squares method by using the second number of data in a range between the start point and the end point, repeating setting processing of the approximate straight line determined by the second number of data while moving the start point and the end point one by one in a direction of the boundary point until the end point reaches the boundary point, setting a first straight line that maximizes K1×W1+1/r when a first constant preset on the basis of a plot interval of adjacent data in the first number of data is denoted by K1, a width from the start point to the end point of the first number of data is denoted by W1, and a gradient of the approximate straight line calculated from the first number of data is denoted by r, setting a second straight line that maximizes K2×W2+1/r when a second constant preset on the basis of a plot interval of the second number of data adjacent on the melting curve is denoted by K2, a width from the start point to the end point of the second number of data is denoted by W2, and a gradient of the approximate straight line calculated from the second number of data is denoted by r, and detecting, as the melting temperature, the temperature of the nucleic acid corresponding to an intersection between the melting curve and a line connecting intermediate values of the absorbance of the nucleic acid at which the temperature of the nucleic acid is the same on the first straight line and the second straight line.

In the detection device and the detection method of the present disclosure, since the melting temperature is univocally determined, it is possible to eliminate a detection error of the melting temperature (Tm value) for each user.

The above and other objects, features, aspects and advantages of the present invention will become apparent from the following detailed description of the present invention to be understood in conjunction with the accompanying drawings.

### BRIEF DESCRIPTION OF THE DRAWINGS

FIG. 1 is a diagram illustrating a configuration of a main part of an analysis device and a detection device.

FIG. 2 is a graph of an ideal melting curve.

FIG. 3 is a graph of a melting curve according to an embodiment.

FIG. 4 is a graph of a melting curve in a case where a data range is set.

FIG. 5 is a graph of a melting curve in a case where the data range is moved.

FIG. 6 is a graph of a melting curve in a case where the data range is reduced.

FIG. 7 is a flowchart of processing of detecting a Tm value.

### DESCRIPTION OF THE PREFERRED EMBODIMENTS

The present embodiment will be described with reference to the drawings. Note that the same or corresponding parts in the drawings are denoted by the same reference signs, and the description thereof will not be repeated.

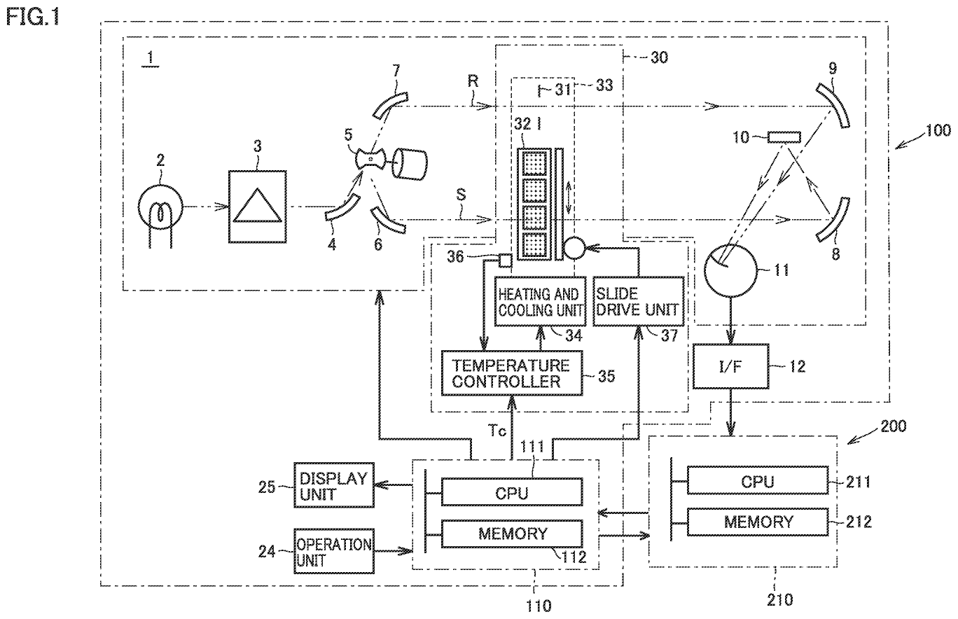

An analysis device 100 to which a detection device 200 according to the embodiment is applied will be described with reference to FIG. 1. FIG. 1 is a diagram illustrating a configuration of a main part of analysis device 100 and detection device 200.

Analysis device 100 includes, as a main part, a photometric unit 1 of a so-called double beam ultraviolet and visible spectrophotometer, a first control device 110 that performs various types of computational processing and control processing, and a sample unit 30 including a multi-cell 32. A predetermined control program is installed in first control device 110, and various types of processing as described later are executed by executing the control program. Detection device 200 is connected to analysis device 100.

First control device 110 includes a central processing unit (CPU) 111 and a memory 112. CPU 111 controls the operation of each unit in photometric unit 1 and sample unit 30 by executing a program stored in memory 112. An operation unit 24 connected to first control device 110 is, for example, a keyboard, a pointing device, or the like, and is used to set various parameters related to measurement and instruct various measurements and processing. A display unit 25 connected to first control device 110 displays auxiliary information for operation, a measurement result, and the like on a screen.

In photometric unit 1, light emitted from a light source 2 is incident on a spectrometer 3, and monochromatic light having a desired wavelength is extracted here. As the wavelength, ultraviolet light within a range of about 230 μm to 280 μm is often used. The monochromatic light is sent to a sector mirror 5 by a reflecting mirror 4, and is divided into two light fluxes of a sample-side light flux S and a reference-side light flux R by sector mirror 5. Sector mirror 5 is provided with a light shield, and a light shielding period is alternately generated with generation periods of sample-side light flux S and reference-side light flux R.

Sample-side light flux S is applied to one cell of multi-cells 32 provided in sample unit 30 via reflecting mirror 6. The light that has passed through one cell of multi-cells 32 is sent to a light receiving surface of a photodetector 11 via reflecting mirrors 8 and 10. On the other hand, reference-side light flux R is emitted to an aperture plate 31 in sample unit 30 via reflecting mirror 7. By passing through aperture plate 31, a light flux diameter on the reference side is aligned with a light flux diameter of the sample side. The light having passed through aperture plate 31 is also sent to the light receiving surface of photodetector 11 via reflecting mirror 9. Note that a dummy cell may be disposed instead of aperture plate 31.

An output signal of photodetector 11 is input to detection device 200 via an interface unit (I/F) 12 including a sample and hold circuit, an analog-digital converter, and the like. Detection device 200 includes a second control device 210. Second control device 210 includes a central processing unit (CPU) 211 and a memory 212. CPU 211 executes various types of computational processing by executing a program stored in memory 212.

CPU 211 executes various types of computational processing for calculating absorbance by using, for example, data from analysis device 100 input to detection device 200 and stored in memory 212. By using the absorbance as a computation result, CPU 211 creates a melting curve that is a graph showing a relationship between a temperature of a nucleic acid and the absorbance. CPU 211 detects a Tm value on the basis of the graph of the melting curve. A specific method of detecting the Tm value will be described later.

In sample unit 30, multi-cell 32 is formed by arranging a plurality of small quartz cells having a capacity of about 10 μl to 100 μl in a line, and the entire multi-cell is configured to reciprocate linearly in a direction substantially orthogonal to sample-side light flux S by a slide drive unit 37. As a result, any cell is selectively irradiated with sample-side light flux S. Multi-cell 32 is held in a thermostatic block 33. Thermostatic block 33 is quickly heated or cooled by a heating and cooling unit 34 using a Peltier element or the like. One or more temperature sensors 36 are attached to thermostatic block 33. The temperature detected by temperature sensor 36 is given to a temperature controller 35. Temperature controller 35 receives a control target value Tc of temperature from CPU 111, and controls power supplied to heating and cooling unit 34 so that the detected temperature becomes control target value Tc, that is, a difference between the temperature and control target value Tc becomes 0. In analysis device 100, by appropriately setting control target value Tc, the absorbance of the sample at an arbitrary temperature in a predetermined temperature range (usually, a range of the capability of heating and cooling unit 34) can be measured.

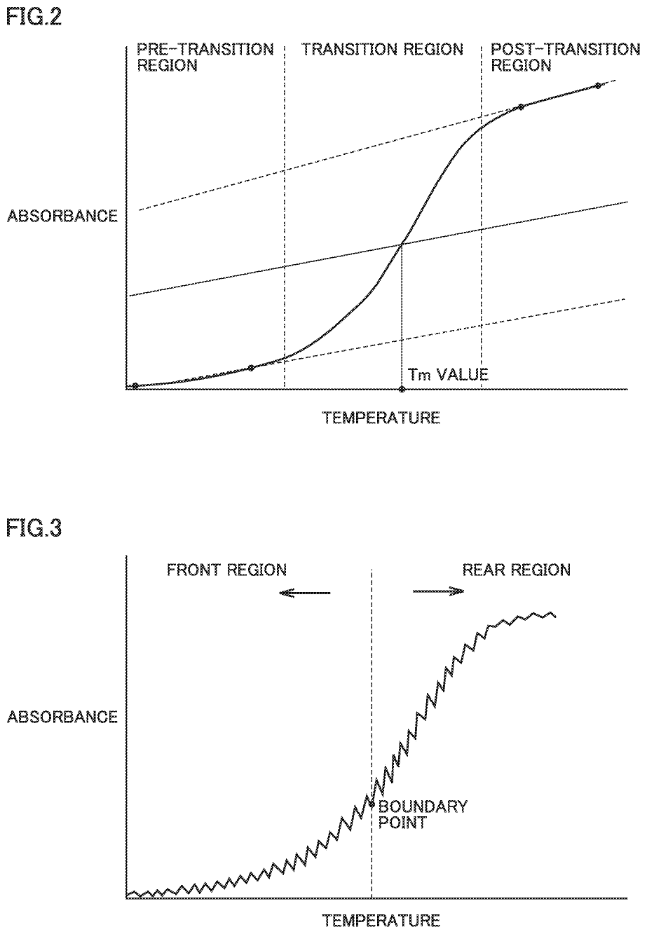

FIG. 2 is a graph of an ideal melting curve. Although noise occurs in the actual graph depending on analysis conditions, a state of the sample, and the like, FIG. 2 shows a graph of an ideal melting curve without noise. In FIG. 2, the horizontal axis represents the temperature of a solution in which the nucleic acid is dissolved, and the vertical axis represents the absorbance of ultraviolet light around a wavelength of 260 μm.

In FIG. 2, a region having a low temperature and a low rate of change in absorbance is referred to as pre-transition region. The pre-transition region is a region in which a double-stranded structure is gradually separated by increasing the temperature of the solution in which the nucleic acid is dissolved. In FIG. 2, a region having a high temperature and a low rate of change in absorbance is referred to as post-transition region. The post-transition region is a region in which most nucleic acids have a single-stranded structure by increasing the temperature of the solution in which the nucleic acid is dissolved. In FIG. 2, a region between the pre-transition region and the post-transition region and having a high rate of change in absorbance is referred to as transition region. The transition region is a region in which the double-stranded structure transitions to the single-stranded structure, and is a region where the absorbance rapidly increases. The temperature at which the proportions of the single-stranded structure and the double-stranded structure in the transition region are equal is a melting temperature (Tm value).

Here, detection of the melting temperature (Tm value) by a conventional method will be described. A user arbitrarily sets a start point and an end point in a portion where a gradient of the graph in the pre-transition region is stable. The user also arbitrarily sets a start point and an end point in a portion where the gradient of the graph in the pre-transition region is stable. In a calculation of the melting temperature (Tm value), there is obtained a line (middle line) connecting intermediate values of absorbance of the nucleic acid at which the temperature of the nucleic acid is the same on a straight line connecting the start point and the end point in the pre-transition region and a straight line connecting the start point and the end point in the post-transition region. The melting temperature (Tm value) is detected as the temperature of the nucleic acid at the intersection of the middle line and the graph of the melting curve.

Here, in the conventional method of detecting the melting temperature (Tm value), the user arbitrarily sets a start point and an end point. Therefore, even when the same data is used, the Tm value varies between the start point and the end point for each user, and thus an error may occur in the detection. In addition, noise occurs in the actual graph depending on the analysis conditions, the state of the sample, and the like. Therefore, when such a graph is used, there is a possibility that an error in the detection of the Tm value increases. Detection device 200 and the detection method using detection device 200 of the present disclosure can eliminate an error for each user in the detection of the Tm value. Hereinafter, the specific method of detecting the Tm value will be described.

FIG. 3 is a graph of the melting curve according to the embodiment. As shown in FIG. 3, the actual graph is a graph in which noise is superimposed on the ideal graph of FIG. 2. The following processing will be described as processing executed by CPU 211 in second control device 210 of detection device 200. CPU 211 determines a boundary point in the graph of the melting curve. The boundary point is a point on the graph set in a region corresponding to the transition region, and is set to, for example, a median value of data of absorbance. A region before the boundary point is defined as a front region, and a region after the boundary point is defined as a rear region.

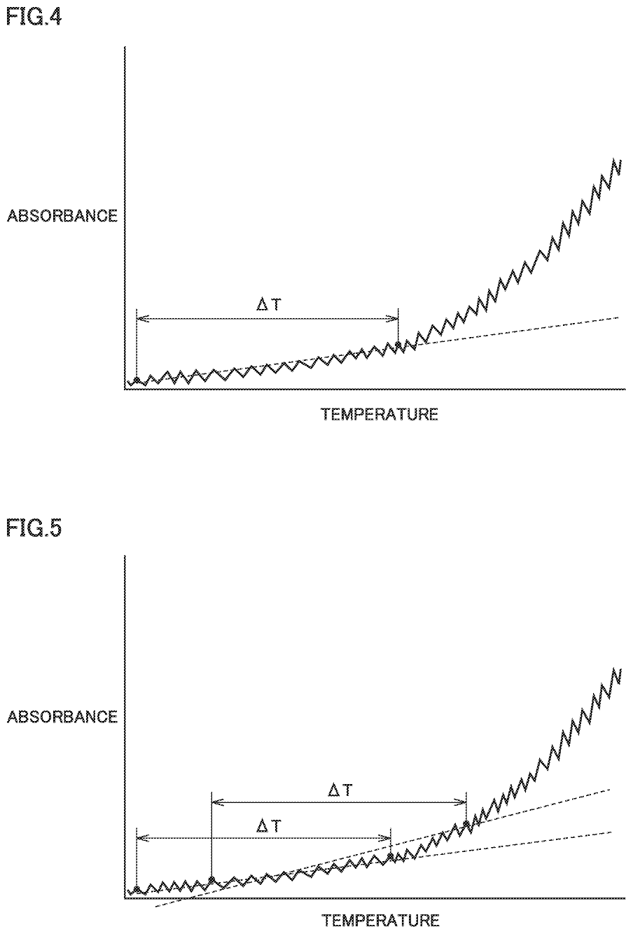

FIG. 4 is a graph of a melting curve in a case where a data range ΔT is set. CPU 211 sets data range ΔT with a minimum temperature as a start point and a maximum temperature as an end point in first data defined by a predetermined number of data in a range of the front region. Data range ΔT is a temperature range that can be arbitrarily set by the user. Data range ΔT may be automatically set by CPU 211. Setting data range ΔT also means setting the number of points of data in a range including the start point and the end point. CPU 211 calculates an approximate straight line by a least squares method by using data in a range including a range between two points at both ends of data range ΔT. The least squares method is a method of obtaining a most probable relational expression by minimizing the sum of squares of errors in processing of a measurement value with an error.

In detection device 200 according to the present embodiment, set data range ΔT is moved and decreased, and setting processing of the approximate straight line is repeated for the moved and decreased data. In detection device 200 according to the present embodiment, one most appropriate approximate straight line among a plurality of obtained approximate straight lines is set in each of the front region and the rear region. As a result, it is possible to set a more appropriate approximate straight line than when an approximate straight line is obtained by simply setting data range ΔT only once. Hereinafter, a case of moving data range ΔT and a case of decreasing data range ΔT will be described by using a graph.

FIG. 5 is a graph of a melting curve in a case where data range ΔT is moved. CPU 211 moves data range ΔT in a direction of the boundary point until the end point reaches the boundary point in the range of the front region. CPU 211 calculates an approximate straight line by the least squares method by using data in moved data range ΔT as a computation target. CPU 211 repeats calculation processing of the approximate straight line until the end point reaches the boundary point. FIG. 5 illustrates a situation of moving from an initial position by a plurality of points. Note that, as the initial position of the start point, a point having the lowest temperature in the graph may be used, or a point moved toward the boundary point by several points may be used. The approximate straight line on the graph in the front region has a characteristic that a gradient of the approximate straight line is greater when the temperature is high than when the temperature is low.

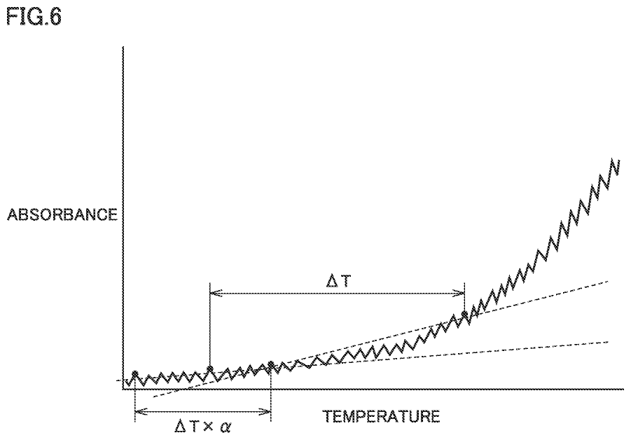

FIG. 6 is a graph of a melting curve in a case where data range ΔT is decreased. After executing the processing of moving the end point to the boundary point and setting the approximate straight line for the data in data range ΔT, CPU 211 similarly executes the processing of setting the approximate straight line for the data in data range ΔT×α in which the number of data points is decreased by multiplying the data in data range ΔT by α that is a predetermined ratio as shown in FIG. 6. α is a numerical value that can be arbitrarily set by the user. For example, the user may decrease the data points in increments of 0.1 (10%) in a range of 0.9 (90%) to 0.2 (20%). When a value obtained by multiplying ΔT by α is not an integer, the number of data points may be set within a range of a value of an integer obtained by rounding down decimal places.

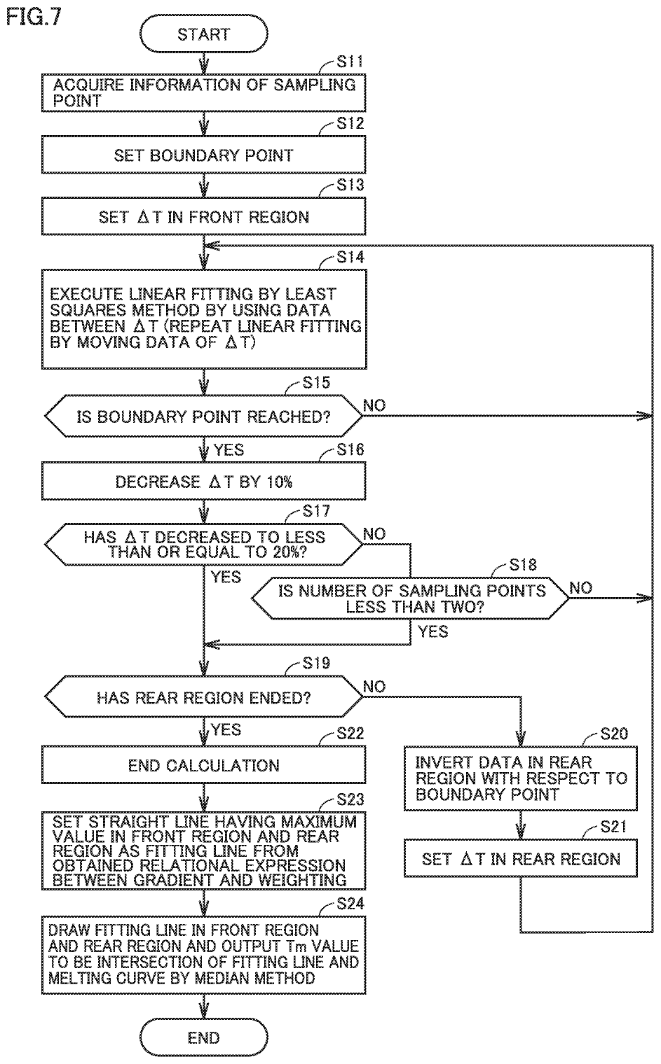

Next, processing of detecting the Tm value will be specifically described. FIG. 7 is a flowchart of the processing of detecting the Tm value. The processing of detecting the Tm value is executed by CPU 211 executing various programs stored in memory 212. Hereinafter, each step in the flowchart is simply referred to as “S”.

In the processing of detecting the Tm value, CPU 211 acquires information of a sampling point (S11). Specifically, CPU 211 calculates the absorbance by using the data input from analysis device 100 to memory 212 of detection device 200, and creates data indicating the relationship between the temperature of the nucleic acid and the calculated absorbance. This data is represented as a melting curve in which the absorbance of the nucleic acid increases as the temperature of the nucleic acid increases when the data is plotted with the temperature of the nucleic acid on the horizontal axis and the absorbance of the nucleic acid on the vertical axis. CPU 211 acquires data of a melting curve created from a plurality of points as information of the sampling point.

Next, CPU 211 sets a boundary point to be a boundary between the front region and the rear region as shown in FIG. 3 (S12). CPU 211 sets a point obtained as a median value of data of the absorbance as a boundary point. Next, CPU 211 sets the data of the temperature range of data range ΔT in the range of the front region as shown in FIG. 4 (S13). The processing of S13 also means setting a minimum temperature as the start point and a maximum temperature as the end point in data range ΔT in the front region, and setting the number of data points in data range ΔT.

Next, as shown in FIG. 4, CPU 211 executes linear fitting by the least squares method by using data between ΔT (S14). While moving the range of data in the direction of the boundary point by one point until the end point reaches the boundary point in S14, CPU 211 repeats the setting processing of the approximate straight line using each data after the moving. As in the case of NO in the processing of S15 and the case of NO in the processing of S18 described later, in a state where the setting processing of the approximate straight line has not been completed, the CPU 211 repeats the processing of S14.

Next, CPU 211 determines whether the end point of data range ΔT has reached the boundary point (S15). When determining that the end point of data range ΔT has not reached the boundary point (NO in S15), CPU 211 executes the processing of S14. When determining that the end point of data range ΔT has reached the boundary point (YES in S15), CPU 211 decreases data range ΔT by 10% (S16). The processing of S16 also means reducing a width of data range ΔT and also means reducing the number of data points. The rate of reduction can be arbitrarily set by the user. When the value obtained by multiplying data range ΔT by the rate of reduction is not an integer, the number of data points may be set within a range of a value of an integer obtained by rounding down decimal places.

Next, CPU 211 determines whether data range ΔT has decreased to less than or equal to 20% (S17). 20% is a lower limit value that can be arbitrarily set by the user. However, if the number of data points is reduced excessively, the influence of an error of the data becomes greater and the detection of an accurate Tm value is hindered. Therefore, the rate of reduction is desirably up to about 20%. When determining that data range ΔT is not decreased to less than or equal to 20% (NO in S17), CPU 211 determines whether the number of sampling points is less than two in S18. That the number of sampling points is less than two (that the number of sampling points is one) means that the setting processing of the linear fitting is not executed. Although it is not normally assumed that the number of sampling points is less than two even if data range ΔT decreases to less than or equal to 20%, the processing of S18 is provided as error processing in a case where some error occurs.

When determining that the number of sampling points is not less than two (NO in S18), CPU 211 repeats the processing of S14. When determining in S17 that data range ΔT has decreased to less than or equal to 20% (YES in S17) or when determining in S18 that the number of sampling points is less than two (YES in S18), CPU 211 proceeds to the processing of S19. In S19, CPU 211 determines whether the processing of setting the linear fitting in the rear region has ended.

When CPU 211 determines that the processing of setting the linear fitting in the rear region has not ended (NO in S19), the data in the rear region is inverted about the boundary point (S20). Here, in the rear region, the maximum temperature in data range ΔT is set as the start point, and the minimum temperature is set as the end point, but in S20, the data is inverted in order to use a processing method of the front region for the processing of the rear region. The processing of S20 is processing of inverting a plot position of the data in the rear region so as to rotate the melting curve point-symmetrically with respect to the boundary point. That is, in the processing of S20, the processing of changing the data so as to invert the data of each sampling point in the rear region corresponding to the temperature of the nucleic acid and the absorbance of the nucleic acid with respect to the boundary point is executed.

Next, CPU 211 sets the data of the temperature range of data range ΔT in the range of the rear region as executed in S13 in FIG. 7 (S21), and proceeds to the processing of S14. The processing of S21 also means setting the maximum temperature as the start point and the minimum temperature as the end point in data range ΔT in the rear region, and setting the number of data points in data range ΔT. In S21, since the data is inverted in S20, the start point is the minimum temperature of data range ΔT and the end point is the maximum temperature of data range ΔT in the actual processing.

CPU 211 executes the processing of inverting the data in the rear region with respect to the boundary point in S20, and thus, the processing of S14 to S18 can be made common, and a processing load can be reduced. The maximum temperature and the minimum temperature in data range ΔT may be set as the start point and the end point, respectively, in the rear region without executing the processing of S20, and while moving data range ΔT to the boundary point until the end point reaches the boundary point, the processing of the linear fitting may be executed by using each data after the moving.

When determining that the setting processing of the linear fitting in the rear region has ended (YES in S19), CPU 211 ends the calculation processing of the linear fitting (S22). Next, CPU 211 sets a straight line having a maximum value in the front region and the rear region as a fitting line from the obtained relational expression between the gradient of the approximate straight line and weighting (S23).

The processing of S23 will be specifically described. A relationship of K=W×C is established when simulation is performed, where W is a plot interval (temperature difference) of adjacent data in the melting curve, a weighting coefficient K is a constant increasing in proportion to a change in W, and C is a constant preset in memory 212. In this manner, weighting coefficient K changes on the basis of the plot interval of data. A weighting coefficient that is a first constant preset on the basis of the plot interval of data in data range ΔT of the front region is denoted as K1, and a weighting coefficient that is a second constant preset on the basis of the plot interval of data in data range ΔT of the rear region is denoted as K2.

In the processing of S23, when the temperature difference between the start point and the end in the front region is W1, and the gradient of the approximate straight line obtained in S14 is r. the approximate straight line in which A1 is maximum at A1=K1/W1+1/r is set to the fitting line in the front region as the first straight line. In the processing of S23, when the temperature difference between the start point and the end in the rear region is W2, and the gradient of the approximate straight line obtained by S14 is r, the approximate straight line in which A2 is maximum at A2=K2×W2+1/r is set to the fitting line in the rear region as the second straight line.

Next, CPU 211 draws the fitting line set in the processing of S23 in the front region and the rear region, detects the temperature of the nucleic acid to be the intersection of the melting curve and the fitting line as the Tm value (melting temperature) by the median method, outputs the Tm value to display unit 25 (S24), and ends the processing.

Here, a specific example of actual measurement of nucleic acid will be described. A reagent M13-25mer is used as the nucleic acid, a concentration of a reagent is 20 μM, an optical path length is 1 mm, and a measurement temperature is from 40° C. to 95° C. When the measurement is performed by analysis device 100 under such conditions, data range ΔT can be set in a range where data range ΔT of the front region is about 30° C. and in a range where data range ΔT of the rear region is about 15° C. In reagent M13-25mer, since the rear region is narrower than the front region, data range ΔT of the rear region is smaller than data range ΔT of the front region. In this manner, data range ΔT changes depending on various conditions. In detection device 200 according to the present embodiment, no matter how data range ΔT is set, the Tm value can be univocally detected by the processing of FIG. 7.

As shown in FIG. 7, in detection device 200, CPU 211 sets one fitting line for each of the front region and the rear region from a plurality of approximate straight lines by the least squares method. CPU 211 detects, as a Tm value (melting temperature), the temperature of the nucleic acid that is an intersection of the melting curve and the fitting line by the median method from the first straight line that is the fitting line in the front region and the second straight line that is the fitting line in the rear region, and outputs the Tm value to display unit 25. In this manner, in detection device 200, since one fitting line is automatically determined in each of the front region and the rear region, it is possible to eliminate a detection error of the melting temperature (Tm value) for each user.

### [Aspects]

It is understood by those skilled in the art that the plurality of exemplary embodiments described above are specific examples of the following aspects.
(Item 1) A detection device according to one aspect relates to a detection device that detects a melting temperature of a nucleic acid by using data indicating a relationship between a temperature of the nucleic acid and absorbance of the nucleic acid corresponding to ultraviolet light, the temperature and the absorbance being measured by an analysis device. The detection device includes a control device and a storage device that stores data from the analysis device. The data is represented by a melting curve in which the absorbance of the nucleic acid increases as the temperature of the nucleic acid increases when the data is plotted with the temperature of the nucleic acid on a horizontal axis and the absorbance of the nucleic acid on a vertical axis. The control device sets a boundary point dividing the data stored in the storage device into a front region and a rear region in accordance with the temperature of the nucleic acid when the data is expressed as the melting curve, sets a minimum temperature as a first start point and a maximum temperature as a first end point on first data defined by a predetermined number of data continuously changing in accordance with the temperature of the nucleic acid among the data in the front region, calculates an approximate straight line by a least squares method by using data in a range between the first start point and the first end point, while moving a range of the first data in a direction of the boundary point until the first end point reaches the boundary point, repeats setting processing of the approximate straight line using each of the first data after the moving, sets a maximum temperature as a second start point and a minimum temperature as a second end point on second data defined by a predetermined number of data continuously changing in accordance with the temperature of the nucleic acid among the data in the rear region, calculates an approximate straight line by the least squares method by using data in a range between the second start point and the second end point, while moving a range of the second data in a direction of the boundary point until the second end point reaches the boundary point, repeats setting processing of the approximate straight line using each of second data after the moving, sets a first straight line that maximizes K1×W1+1/r when a first constant preset on the basis of a plot interval of adjacent data in the first data is denoted by K1, a temperature difference between the first start point and the first end point of the first data is denoted by W1, and a gradient of the approximate straight line calculated from the first data is denoted by r, sets a second straight line that maximizes K2×W2+1/r when a second constant preset on the basis of a plot interval of adjacent data in the second data is denoted by K2, a temperature difference between the second start point and the second end point of the second data is denoted by W2, and a gradient of the approximate straight line calculated from the second data is denoted by r, and detects, as the melting temperature, the temperature of the nucleic acid corresponding to an intersection between the melting curve and a line connecting intermediate values of the absorbance of the nucleic acid at which the temperature of the nucleic acid is the same on the first straight line and the second straight line.

In the detection device according to item 1, since the melting temperature is univocally detected, it is possible to eliminate a detection error of the melting temperature for each user.
(Item 2) In the detection device according to item 1, the control device repeats setting processing of the first straight line by decreasing the number of data of the first data by a predetermined number in the front region and changing W1 in accordance with the number of data decreased, repeats setting processing of the second straight line by decreasing the number of data of the second data by a predetermined number in the rear region and changing W2 in accordance with the number of data decreased, and detects, as the melting temperature, the temperature of the nucleic acid corresponding to the intersection between the melting curve and the line connecting the intermediate values of the absorbance of the nucleic acid at which the temperature of the nucleic acid is the same on the first straight line and the second straight line.

In the detection device according to item 2, since the first straight line and the second straight line is set after the number of data is decreased by a predetermined number, it is possible to eliminate the detection error of the melting temperature.
(Item 3) In the detection device according to item 1 or 2, the control device ends the setting processing of the first straight line when the number of data of the first data reaches a predetermined lower limit value, and ends the setting processing of the second straight line when the number of data of the second data reaches a predetermined lower limit value.

In the detection device according to item 3, since the lower limit value is determined, it is possible to prevent use of data in an unnecessary range in which the detection error increases.
(Item 4) In the detection device according to any one of items 1 to 3, the control device executes the setting processing of the second straight line by inverting a plot position of the data in the rear region so as to rotate the melting curve in point symmetry with respect to the boundary point, and then, inverts the plot position set of the data of the second straight line again with respect to the boundary point.

In the detection device according to item 4, since the rear region can be processed similarly to the front region, a processing load can be reduced.
(Item 5) A detection method according to one aspect relates to a method of detecting a melting temperature of a nucleic acid by using data indicating a relationship between a temperature of the nucleic acid and absorbance of the nucleic acid corresponding to ultraviolet light, the temperature and the absorbance being measured by an analysis device. The data is represented by a melting curve in which the absorbance of the nucleic acid increases as the temperature of the nucleic acid increases when the data is plotted with the temperature of the nucleic acid on a horizontal axis and the absorbance of the nucleic acid on a vertical axis. The method includes executing steps of recording data from an analysis device, setting a boundary point dividing the data stored into a front region and a rear region in accordance with the temperature of the nucleic acid when the data is expressed as the melting curve, setting a start point and an end point in order from a point at which the temperature of the nucleic acid is lower on the melting curve on a first number of data continuously changing in accordance with the temperature of the nucleic acid among the data in the front region, calculating an approximate straight line by a least squares method by using the first number of data in a range between the first start point and the first end point, repeating setting processing of the approximate straight line from the first number of data while moving the start point and the end point one by one in a direction of the boundary point until the end point reaches the boundary point, setting a start point and an end point in order from a point at which the temperature of the nucleic acid is higher on a second number of data continuously changing in accordance with the temperature of the nucleic acid among the data in the rear region, calculating an approximate straight line by the least squares method by using the second number of data in a range between the start point and the end point, repeating setting processing of the approximate straight line determined by the second number of data while moving the start point and the end point one by one in a direction of the boundary point until the end point reaches the boundary point, setting a first straight line that maximizes K1×W1+1/r when a first constant preset on the basis of a plot interval of adjacent data in the first number of data is denoted by K1, a width from the start point to the end point of the first number of data is denoted by W1, and a gradient of the approximate straight line calculated from the first number of data is denoted by r, setting a second straight line that maximizes K2×W2+1/r when a second constant preset on the basis of a plot interval of the second number of data adjacent on the melting curve is denoted by K2, a width from the start point to the end point of the second number of data is denoted by W2, and a gradient of the approximate straight line calculated from the second number of data is denoted by r, and detecting, as the melting temperature, the temperature of the nucleic acid corresponding to an intersection between the melting curve and a line connecting intermediate values of the absorbance of the nucleic acid at which the temperature of the nucleic acid is the same on the first straight line and the second straight line.

In the detection method according to item 5, since the melting temperature is univocally detected, it is possible to eliminate a detection error of the melting temperature for each user.
(Item 6) The detection method according to item 5 includes executing steps of repeating setting processing of the first straight line by decreasing the width of the first number of data by a predetermined ratio in the front region and changing W1 in accordance with the width decreased, repeating setting processing of the second straight line by decreasing the width of the second number of data by a predetermined ratio in the rear region and changing W2 in accordance with the width decreased, and detecting, as the melting temperature, the temperature of the nucleic acid corresponding to the intersection between the melting curve and the line connecting the intermediate values of the absorbance of the nucleic acid at which the temperature of the nucleic acid is the same on the first straight line and the second straight line.

In the detection method according to item 6, since the first straight line and the second straight line is set after the width is decreased by a predetermined ratio, it is possible to eliminate the detection error of the melting temperature.
(Item 7) The detection method according to item 5 or 6 further includes executing steps of ending the setting processing of the first straight line when the number of plots of the first number of data reaches a predetermined lower limit value, and ending the setting processing of the second straight line when the number of plots of the second number of data reaches a predetermined lower limit value.

In the detection method according to item 7, since the lower limit value is determined, it is possible to prevent use of data in an unnecessary range in which the detection error increases.
(Item 8) The detection method according to any one of items 5 to 7 includes executing a step of executing the setting processing of the second straight line by inverting a plot position of the data in the rear region so as to rotate the melting curve in point symmetry with respect to the boundary point, and then, inverting the plot position set of the data of the second straight line again with respect to the boundary point.

In the detection method according to item 8, since the rear region can be processed similarly to the front region, a processing load can be reduced.

Although the embodiment of the present invention has been described above, it should be understood that the embodiment disclosed herein is illustrative in all respects and not restrictive. The scope of the present invention is indicated by the claims, and it is intended that all modifications within the meaning and scope of the claims are included in the present invention.

What is claimed is:

1. A detection device that detects a melting temperature of a nucleic acid by using data indicating a relationship between a temperature of the nucleic acid and an absorbance of the nucleic acid corresponding to ultraviolet light, the temperature and the absorbance being measured by an analysis device, the detection device comprising:
a control device; and
a storage device that stores the data from the analysis device,
wherein the data is represented by a melting curve in which the absorbance of the nucleic acid increases as the temperature of the nucleic acid increases when the data is plotted with the temperature of the nucleic acid on a horizontal axis and the absorbance of the nucleic acid on a vertical axis, and
the control device
sets a boundary point dividing the data stored in the storage device into a front region and a rear region in accordance with the temperature of the nucleic acid when the data is expressed as the melting curve,
sets a minimum temperature as a first start point and a maximum temperature as a first end point on first data defined by a predetermined number of data continuously changing in accordance with the temperature of the nucleic acid among the data in the front region,
calculates an approximate straight line by a least squares method by using data in a range between the first start point and the first end point,
while moving a range of the first data in a direction of the boundary point until the first end point reaches the boundary point, repeats setting processing of the approximate straight line using each of the first data after the moving,
sets a maximum temperature as a second start point and a minimum temperature as a second end point on second data defined by a predetermined number of data continuously changing in accordance with the temperature of the nucleic acid among the data in the rear region,
calculates an approximate straight line by the least squares method by using data in a range between the second start point and the second end point,
while moving a range of the second data in a direction of the boundary point until the second end point reaches the boundary point, repeats setting processing of the approximate straight line using each of the second data after the moving,
sets a first straight line that maximizes K1×W1+1/r when a first constant preset on the basis of a plot interval of adjacent data in the first data is denoted by K1, a temperature difference between the first start point and the first end point of the first data is denoted by W1, and a gradient of the approximate straight line calculated from the first data is denoted by r,
sets a second straight line that maximizes K2×W2+1/r when a second constant preset on the basis of a plot interval of adjacent data in the second data is denoted by K2, a temperature difference between the second start point and the second end point of the second data is denoted by W2, and a gradient of the approximate straight line calculated from the second data is denoted by r, and
detects, as the melting temperature, the temperature of the nucleic acid corresponding to an intersection between the melting curve and a line connecting intermediate values of the absorbance of the nucleic acid at which the temperature of the nucleic acid is the same on the first straight line and the second straight line.

2. The detection device according to claim 1, wherein the control device
repeats setting processing of the first straight line by decreasing the number of data of the first data by a predetermined number in the front region and changing W1 in accordance with the number of data decreased,
repeats setting processing of the second straight line by decreasing the number of data of the second data by a predetermined number in the rear region and changing W2 in accordance with the number of data decreased, and
detects, as the melting temperature, the temperature of the nucleic acid corresponding to the intersection between the melting curve and the line connecting the intermediate values of the absorbance of the nucleic acid at which the temperature of the nucleic acid is the same on the first straight line and the second straight line.

3. The detection device according to claim 2, wherein the control device
ends the setting processing of the first straight line when the number of data of the first data reaches a predetermined lower limit value, and
ends the setting processing of the second straight line when the number of data of the second data reaches a predetermined lower limit value.

4. The detection device according to claim 1, wherein the control device
executes the setting processing of the second straight line by inverting a plot position of the data in the rear region so as to rotate the melting curve in point symmetry with respect to the boundary point, and then,
inverts the plot position set of the data of the second straight line again with respect to the boundary point.

5. A method of detecting a melting temperature of a nucleic acid by using data indicating a relationship between a temperature of the nucleic acid and an absorbance of the nucleic acid corresponding to ultraviolet light, the temperature and the absorbance being measured by an analysis device,
wherein the data is represented by a melting curve in which the absorbance of the nucleic acid increases as the temperature of the nucleic acid increases when the data is plotted with the temperature of the nucleic acid on a horizontal axis and the absorbance of the nucleic acid on a vertical axis, and
the method comprises executing steps of:
setting a boundary point dividing the data stored into a front region and a rear region in accordance with the temperature of the nucleic acid when the data is expressed as the melting curve;
setting a minimum temperature as a first start point and a maximum temperature as a first end point on first data defined by a predetermined number of data continuously changing in accordance with the temperature of the nucleic acid among the data in the front region;
calculating an approximate straight line by a least squares method by using data in a range between the first start point and the first end point;
while moving a range of the first data in a direction of the boundary point until the first end point reaches the boundary point, repeating setting processing of the approximate straight line using each of the first data after the moving;
setting a maximum temperature as a second start point and a minimum temperature as a second end point on second data defined by a predetermined number of data continuously changing in accordance with the temperature of the nucleic acid among the data in the rear region;
calculating an approximate straight line by the least squares method by using data in a range between the second start point and the second end point;
while moving a range of the second data in a direction of the boundary point until the second end point reaches the boundary point, repeating setting processing of the approximate straight line using each of the second data after the moving;
setting a first straight line that maximizes K1×W1+1/r when a first constant preset on the basis of a plot interval of adjacent data in the first data is denoted by K1, a temperature difference between the first start point and the first end point of the first data is denoted by W1, and a gradient of the approximate straight line calculated from the first data is denoted by r;
setting a second straight line that maximizes K2×W2+1/r when a second constant preset on the basis of a plot interval of adjacent data in the second data is denoted by K2, a temperature difference between the second start point and the second end point of the second data is denoted by W2, and a gradient of the approximate straight line calculated from the second data is denoted by r; and
detecting, as the melting temperature, the temperature of the nucleic acid corresponding to an intersection between the melting curve and a line connecting intermediate values of the absorbance of the nucleic acid at which the temperature of the nucleic acid is the same on the first straight line and the second straight line.

6. The method according to claim 5, further comprising executing steps of:
repeating setting processing of the first straight line by decreasing the number of data of the first data by a predetermined number in the front region and changing W1 in accordance with the number of data decreased;
repeating setting processing of the second straight line by decreasing the number of data of the second data by a predetermined number in the rear region and changing W2 in accordance with the number of data decreased; and
detecting, as the melting temperature, the temperature of the nucleic acid corresponding to the intersection between the melting curve and the line connecting the intermediate values of the absorbance of the nucleic acid at which the temperature of the nucleic acid is the same on the first straight line and the second straight line.

7. The method according to claim 6, further comprising executing steps of:
ending the setting processing of the first straight line when the number of data of the first data reaches a predetermined lower limit value; and
ending the setting processing of the second straight line when the number of data of the second data reaches a predetermined lower limit value.

8. The method according to claim 5, further comprising executing a step of executing the setting processing of the second straight line by inverting a plot position of the data in the rear region so as to rotate the melting curve in point symmetry with respect to the boundary point, and then, inverting the plot position set of the data of the second straight line again with respect to the boundary point.
